(19) United States
(12) Patent Application Publication
(10) Pub. No.: US 2025/0051723 A1
(43) Pub. Date: Feb. 13, 2025

(54) POURABLE PLACENTAL COLLAGEN MATRIX SHEET

(71) Applicant: MiMedx Group, Inc., Marietta, GA (US)

(72) Inventors: Michelle Massee, Milton, GA (US); Jake Mullan, Atlanta, GA (US); Rick LeVaughn, Marietta, GA (US); Shauna Campbell, Marietta, GA (US)

(21) Appl. No.: 18/798,070

(22) Filed: Aug. 8, 2024

Related U.S. Application Data
(60) Provisional application No. 63/518,286, filed on Aug. 8, 2023.

(51) Int. Cl. C12N 5/073

(52) U.S. Cl. CPC C12N 5/0605

(57) ABSTRACT

A placental collagen matrix (PCM) that can be poured into a customized tray shape and then dehydrated that can be used in the treatment of wounds. Specifically, the PCM has the property of being able to be poured into a variety of shapes, sizes and thickness. The resulting customized graft may be used in the treatment of a variety of wounds. The PCM may or may not be decellularized, and may be made of placental disc, umbilical cord, amnion, chorion, and/or intermediate layer.

### CROSS-REFERENCE TO RELATED APPLICATIONS

This application claims the benefit of U.S. Provisional Application No. 63/518,286, filed Aug. 8, 2023, the contents of which are hereby incorporated by reference in their entirety.

### FIELD OF THE INVENTION

The invention relates to a poured, decellularized, placental tissue-derived extracellular matrix that may be configured into custom shapes and sizes, dependent upon the size and shape of the mold that the matrix is poured into.

### BACKGROUND

Human placental membrane (e.g., amniotic membrane or tissue) has been used for various types of reconstructive surgical procedures since the early 1900s. The membrane serves as a substrate material, more commonly referred to as a biological dressing or patch graft. Such membrane has also been used widely for ophthalmic procedures in the United States and in countries in the southern hemisphere. Typically, such membrane is either frozen or dried for preservation and storage until needed for surgery.

Such placental tissue is typically harvested after an elective Cesarean surgery. Extracellular matrix derived from such placental tissue provides unique grafting characteristics when used for surgical procedures, including providing a matrix for cellular migration/proliferation and a natural biological barrier. Extracellular matrix grafts are non-immunogenic, promote increased self-healing, are susceptible of being fixed in place using different techniques including fibrin glue or suturing. And, such grafts, when properly prepared, can be stored at room temperature for extended periods of time, without need for refrigeration or freezing, until needed for a surgical procedure.

Known clinical procedures or applications for such placental membrane grafts include Schneiderian Membrane repair (i.e., sinus lift), guided tissue regeneration (GTR), general wound care, and primary closure membrane. Known clinical procedures or applications for such placental membrane grafts also include biological wound dressing.

A detailed look at the history and procedure for harvesting and using “live” amniotic tissue for surgical procedures and a method for harvesting and freezing amniotic tissue grafts for ophthalmic procedures is described in U.S. Pat. No. 6,152,142 issued to Tseng, which is incorporated herein by reference in its entirety.

However, the use of placental membrane as a graft may also present certain drawbacks. For example, placental tissue-derived grafts are generally limited to the dimensions of the source tissue when it comes to the length, width and thickness of the end product. After processing, amnion and chorion layers are approximately 100 μm in thickness. Such thin grafts are effective for a variety of treatments. However, there are applications where a thicker graft would be advantageous. If placental tissue is the source material, a thicker graft can be obtained by sandwiching multiple placental tissues into successive layers. Width and length can also be adjusted, but the maximum length and width will always be constrained by the size of the source tissue. Additionally, repositioning of an amniotic graft after hydration is often difficult, as it tends to fold over on itself, making it less suitable for certain surgical applications. Accordingly, it would be advantageous to develop a graft that can be customized into different shapes and thicknesses that are not dependent upon the size and dimensions of the source placental tissue. Accordingly, one alternative is to use common decellularization techniques to convert placental tissue into a placental collagen matrix (PCM) that may be poured into custom shapes of various dimensions that are not limited by the dimensions of the original source tissue.

There are a number of commercially available products currently on the market that are derived from extracellular matrix (ECM); however, they are derived from xenogeneic sources. Xenografts require thorough decellularization to remove any species-specific markers and antigenic components. Often, these methods remove almost all components of the tissue, leaving behind only the structural elements. Whereas with PCM, the processing is optimized to decellularize the tissue, while retaining matrix-bound regulatory factors. These regulatory factors are human in origin; therefore, they are capable of stimulating a response from a human recipient if the protein conformation is kept intact during the tissue preservation process.

As previously noted, of the available products that are derived from human placental tissue, most, if not all, can be classified as thin grafts or particulate matrices. A particulate matrix is beneficial for covering larger areas; however, it lacks the structural stability and handling characteristics of a sheet configuration, and can be difficult to apply due to static electricity buildup, which may cause the particulate matrix to clump and adhere to the wall of the vial in which it is stored. Particulate matrices can be hydrated to form a “paste” for easier application, but this still negates many of the structural benefits of a sheet configuration. Particulate and thin grafts can easily “float” out of a target area due to their smaller size/thickness, whereas a thicker, more easily conforming sheet would be much harder to move from that same area, and is more likely to adhere to the wound site. Accordingly, a composition that combines the structural integrity of a sheet product with the versatility of a particulate matrix is desirable.

### SUMMARY OF THE INVENTION

In certain embodiments, the subject matter described herein is obtained by applying commonly known decellularization and dehydration techniques to human placental disc tissue. The decellularized, dehydrated placental disc tissue can then be morselized into a PCM, which shares certain characteristics of both sheet and particulate matrix product configurations. While this application is directed toward the use of PCM, the process can be modified to use alternative tissue source material, such as umbilical cord, amnion and/or chorion tissue and is not limited solely to PCM as a source material of tissue.

After harvesting, the placental disc tissue is treated in a number of steps to provide the products described herein. For example, the placental disc tissue is separated from other placental tissues, such as the umbilical cord and amniotic membrane and the chorion layers (the other placental tissues aside from the disc may be retained for other purposes). All components are sourced from a single donor.

After the placental disc tissue has been separated and isolated from the other placental tissues, it is subjected to a wash in sterile water. After the disc tissue has been rinsed in the sterile water, it is then strained and placed in a blender.

After the disc tissue has been blended to the desired consistency, additional sterile water is added to the blended tissue. Once the blended disc tissue has been rinsed, it is then subjected to a decellularization process. Many decellularization processes are known to those skilled in the art, and any can be utilized during this step of the process. However, for the sake of clarity, a specific decellularization process will be described in further detail.

Once the decellularization process has been completed, the decellularized placental disc tissue is subjected to additional water rinses, and then poured into a beaker so that its volume may be recorded. Once the volume of decellularized tissue has been recorded, the decellularized tissue is subjected to another blending step.

When the decellularized tissue has been appropriately blended, more sterile water is added to the mixture. Once the water has been incorporated into the decellularized tissue solution, it can then be poured into a tray having any dimension (shape/thickness). Once the decellularized tissue solution has been poured into the tray, it is then sealed in a Tyvek pouch. After being sealed in the pouch, the tray, tissue and Tyvek pouch are placed into a commercial lyophilizer and freeze dried to the desired level of residual moisture.

Upon completion of the lyophilization cycle, the decellularized tissue sheet is removed from the lyophilizer and inspected for uniformity and integrity. If the decellularized tissue sheet is of acceptable uniformity and integrity, it can either be packaged or further cut to size (if further cutting is desired).

### BRIEF DESCRIPTION OF THE FIGURES

Further features and benefits of the present invention will be apparent from a detailed description of preferred embodiments thereof taken in conjunction with the following drawings, wherein similar elements are referred to with similar reference numbers, and wherein:

FIG. 1 is a photograph of a decellularized sheet that was poured into a tray shaped in the form of a human face.

FIG. 2 is a photograph of a decellularized sheet that was poured into a tray shaped in the form of human hand.

FIG. 3 is a photograph of a decellularized sheet that was poured into a tray in the shape of a rectangular sheet.

### DETAILED DESCRIPTION OF THE INVENTION

One challenge that has been encountered in the field is that placental tissue grafts are constrained in size and shape by the source tissue. Additionally, amnion and chorion tissues tend to be very thin and fragile, and may tear or deform when utilized in procedures where the graft may be exposed to even minimal torsional and/or shear forces.

Accordingly, there is a need in the marketplace for a graft that can be made into a variety of shapes, and that is sturdier and more durable than amnion and chorion-derived placental tissue grafts.

It is to be understood that this invention is not limited to particular embodiments described, as such may, of course, vary. It is also to be understood that the terminology used herein is for the purpose of describing particular embodiments only, and is not intended to be limiting, since the scope of this invention will be limited only by the appended claims.

The detailed description of the invention is divided into various sections only for the reader's convenience and disclosure found in any section may be combined with that in another section. Unless defined otherwise, all technical and scientific terms used herein have the same meaning as commonly understood by one of ordinary skill in the art to which this invention belongs. Although any methods and materials similar or equivalent to those described herein can also be used in the manufacture, practice or testing of the present invention, the preferred methods and materials are now described. All patents and publications mentioned herein are incorporated by reference to disclose and describe the methods and/or materials in connection with which the publications are cited.

Each embodiment disclosed herein is contemplated as being applicable to each of the other disclosed embodiments. All combinations and sub-combinations of the various elements described herein are within the scope of the embodiments.

It is understood that where a parameter range is provided, all integers and ranges within that range, and tenths and hundredths thereof, are also provided by the embodiments. For example, “5-10%” includes 5%, 6%, 7%, 8%, 9%, and 10%; 5.0%, 5.1%, 5.2% . . . 9.8%, 9.9%, and 10.0%; and 5.00%, 5.01%, 5.02% . . . 9.98%, 9.99%, and 10.00%, as well as, for example, 6-9%, 5.1%-9.9%, and 5.01%-9.99%. This also applies to ratios. For example, a recited ratio range of “1:100 to 200:1” includes ratios such as 1:50, 1:1, and 100:1, along with ranges such as 1:100 to 1:1, 1:50 to 50:1, and 1:1 to 200:1.

As used herein, “about” in the context of a numerical value or range means within ±1%, ±5%, or 10% of the numerical value or range recited or claimed.

The invention illustratively disclosed herein suitably may be practiced in the absence of any element which is not specifically disclosed herein.

It must be noted that as used herein and in the appended claims, the singular forms “a”, “an”, and “the” include plural referents unless the context clearly dictates otherwise.

### Definitions

As used herein the following terms have the following meanings.

“Placental tissue” or “placenta” means the umbilical cord, the placental disc, and the amniotic sac, which consists of the amnion, intermediate spongy layer, and the chorion.

“Placental Disc” means the section of the placenta composed of the fetal portion known as the chorionic plate and the maternal portion known as the basal plate decidua.

“Placental Collagen Matrix (PCM)” means placental disc tissue that has been decellularized.

“Comprising” or “comprises” is intended to mean that the compositions, for example media, and methods include the recited elements, but not excluding others. “Consisting essentially of” when used to define methods, shall mean excluding other elements of any essential significance to the combination for the stated purpose. “Consisting of” shall mean excluding additional substantial method steps. Embodiments defined by each of these transition terms are within the scope of this invention. In other words, if an embodiment using the term “comprising” is explicitly recited, otherwise identical embodiments using the term “consisting of” or “consisting essentially of” are also within the scope of the invention.

“Dehydrated” means that the tissue has had substantially all of its water removed, (i.e. greater than 85%, greater than 90%, greater than 95%, greater than 99%, or 100% of its water removed).

“Optional” or “optionally” means that the subsequently described event or circumstance can or cannot occur, and that the description includes instances where the event or circumstance occurs and instances where it does not.

The term “subject” as used herein is any vertebrate organism including but not limited to mammalian subjects such as humans, farm animals, domesticated pets and the like. The term “patient” may be used interchangeably with “subject.”

The term “treat,” with respect to a wound, means to reduce the amount of time the wound would have taken to heal in the absence of any type of medical intervention.

### Detailed Description

An embodiment of the invention is a flowable composition, comprising decellularized, morselized placental disc and a carrier.

An embodiment of the invention is a flowable composition, comprising morselized umbilical cord tissue and a carrier.

An embodiment of the invention is a flowable composition, comprising morselized amnion, chorion, or intermediate layer, or any combination of these tissue types.

In an embodiment, the composition has a viscosity of at most about 100,000 cP.

In an embodiment, the composition has a viscosity of between about 0.89 cP and about 100,000 cP. In an embodiment, the composition has a viscosity of at least, at most, or about 0.89 cP, 5 cP, 10 cP, 50 cP, 100 cP, 500 cP, 1,000 cP, 5,000 cP, 10,000 cP, 20,000 cP, 30,000 cP, 40,000 cP, 50,000 cP, 60,000 cP, 70,000 cP, 80,000 cP, 90,000 cP, or 100,000 cP, or within a range defined by any two of these values.

In an embodiment, the carrier is sterile water.

In an embodiment, the composition comprises about 70 to about 30 weight percent placental disc and about 30 to about 70 weight percent carrier. In an embodiment, the composition comprises about 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, or 30 weight percent placental disc, or within a range defined by any two of these values. In an embodiment, the composition comprises about 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 weight percent carrier.

In an embodiment, the composition comprises about 70 to about 30 weight percent umbilical cord and about 30 to about 70 weight percent carrier. In an embodiment, the composition comprises about 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, or 30 weight percent umbilical cord, or within a range defined by any two of these values. In an embodiment, the composition comprises about 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 weight percent carrier.

In an embodiment, the composition comprises about 70 to about 30 weight percent amnion and/or chorion and about 30 to about 70 weight percent carrier. In an embodiment, the composition comprises about 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, or 30 weight percent amnion and/or chorion, or within a range defined by any two of these values. In an embodiment, the composition comprises about 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 weight percent carrier.

In an embodiment, the composition does not comprise amnion, chorion, or umbilical cord.

In an embodiment, the composition does not comprise placental disc.

In an embodiment, the composition consists essentially of decellularized, morselized placental disc and a carrier. In an embodiment, the composition consists of decellularized, morselized placental disc and a carrier.

In an embodiment, the composition consists essentially of morselized amnion, chorion, and/or umbilical cord and a carrier. In an embodiment, the composition consists of morselized amnion, chorion, and/or umbilical cord and a carrier.

In an embodiment, the morselized tissue has a particle size of at least, at most, or about 1, 2, 5, 10, 20, 30, 40, 50, 60, 70, 80, 90, 100, 125, 150, 175, 200, 225, 250, 275, 300, 350, 400, 450, 500, 600, 700, 800, 900, or 1000 microns, or within a range defined by any two of these values.

Another embodiment is a dehydrated sheet comprising decellularized, morselized placental disc. An alternative embodiment is a dehydrated sheet comprising morselized umbilical cord. An alternative embodiment is a dehydrated sheet comprising morselized amnion and/or chorion.

In an embodiment, the sheet has a thickness of at least about 0.05 mm.

In an embodiment, the sheet has a thickness of between about 0.05 mm and about 40 mm. In an embodiment, the sheet has a thickness of at least, at most, or about 0.05, 0.1, 0.2, 0.3, 0.4, 0.5, 0.6, 0.7, 0.8, 0.9, 1, 1.5, 2, 2.5, 3, 3.5, 4, 4.5, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 mm, or within a range defined by any two of these values.

In an embodiment, the sheet comprises a first side and a second side, and the first side has a surface area of at least about 0.5 cm².

In an embodiment, the surface area of said first side is between about 0.5 cm² and about 800 cm². In an embodiment, the surface area of said first side is at least, at most, or about 0.5, 1, 2, 3, 4, 5, 6, 7, 8, 9, 10, 11, 12, 13, 14, 15, 16, 17, 18, 19, 20, 25, 30, 40, 50, 60, 70, 80, 90, 100, 150, 200, 250, 300, 350, 400, 450, 500, 550, 600, 650, 700, 750, or 800 cm², or within a range defined by any two of these values.

In an embodiment, the sheet has not been treated with a crosslinking agent.

In an embodiment, the sheet consists essentially of decellularized, morselized placental disc. In an embodiment, the sheet consists of decellularized, morselized placental disc.

In an embodiment, the sheet consists essentially of morselized umbilical cord. In an embodiment, the sheet consists of decellularized, morselized umbilical cord.

In an embodiment, the sheet consists essentially of morselized amnion and/or chorion. In an embodiment, the sheet consists of morselized amnion and/or chorion.

An embodiment is a sealed container comprising a sheet as described above.

In an embodiment, the container is deoxygenated. In an embodiment, the container is a sealed pouch.

An embodiment is a method of preparing a decellularized, morselized placental disc composition, said method comprising:
i) morselizing placental disc; and
ii) decellularizing the morselized placental disc obtained in step i).

In an embodiment, the method further comprises an additional morselization step following step ii).

In an embodiment, the composition further comprises a carrier.

In an embodiment, the method further comprises
iii) transferring the decellularized, morselized placental disc composition into a container having a desired shape; and
iv) dehydrating the composition in the container,
   thereby obtaining a dehydrated, decellularized, morselized placental disc sheet.

An embodiment is a method of preparing a dehydrated, morselized umbilical cord sheet, said method comprising:
i) morselizing umbilical cord, thereby forming a morselized umbilical cord composition;
ii) transferring the morselized umbilical cord composition into a container having a desired shape; and
iii) dehydrating the composition in the container,
   thereby obtaining a dehydrated, morselized umbilical cord sheet.

In an embodiment, the composition further comprises a carrier.

An embodiment is a method of preparing a dehydrated, morselized amnion and/or chorion sheet, said method comprising:
i) morselizing amnion and/or chorion, thereby forming a morselized amnion and/or chorion composition;
ii) transferring the morselized amnion and/or chorion composition into a container having a desired shape; and
iii) dehydrating the composition in the container,
   thereby obtaining a dehydrated, morselized amnion and/or chorion sheet.

In an embodiment, the composition further comprises a carrier.

In an embodiment, said transferring is via pouring and said container is a tray or mold. In an embodiment, said transferring is via injection and said container is a mold.

In an embodiment, the placental disc tissue is dehydrated. In an embodiment, the placental disc tissue is lyophilized.

In an embodiment, the umbilical cord tissue is dehydrated. In an embodiment, the umbilical cord tissue is lyophilized.

In an embodiment, the amnion and/or chorion is dehydrated. In an embodiment, the amnion and/or chorion is lyophilized.

An embodiment of the invention is also a method of covering or treating a wound comprising, contacting a wound with a sheet as described herein, optionally having a desired shape, size and/or thickness as described herein.

### Methods of Manufacture

Human placentas that meet the above selection criteria are preferably individually bagged in a saline solution in a sterile shipment bag and stored in a container of wet ice for shipment to a processing location or laboratory for further processing.

### Initial Tissue Collection

The recovery of placenta tissue originates in a hospital, where it is collected from a healthy donor after giving birth. The donor, referring to the mother who is about to give birth, voluntarily submits to a comprehensive screening process designed to provide the safest tissue possible for transplantation. The screening process preferably tests for antibodies to the human immunodeficiency virus type 1 and type 2 (anti-HIV-1 and anti-HIV-2), hepatitis B surface antigens (HBsAg), antibodies to the hepatitis C virus (anti-HCV), antibodies to the human T-lymphotropic virus type I and type II (anti-HTLV-I and anti-HTLV-II), CMV, and syphilis, using conventional serological tests. The above list of tests is exemplary only, as more, fewer, or different tests may be desired or necessary over time or based upon the intended use of the grafts, as will be appreciated by those skilled in the art.

Based upon a review of the donor's information and screening test results, the donor will either be deemed acceptable or not. In addition, at the time of delivery, cultures are taken to determine the presence of, for example, Clostridium or Streptococcus. If the donor's information, screening tests, and the delivery cultures are all negative (i.e. do not indicate any risks or indicate acceptable level of risk), the donor is approved and the tissue specimen is designated as initially eligible for further processing and evaluation.

Human placentas that meet the above selection criteria are preferably individually bagged in a saline solution in a sterile shipment bag and stored in a container of wet ice for shipment to a processing location or laboratory for further processing.

### Material Check-In and Evaluation

Upon arrival at the processing center or laboratory, the shipment is opened and it is verified that the sterile shipment bag/container is still sealed and intact, that ice or other coolant is present and that the contents are cool, that the appropriate donor paperwork is present, and that the donor number on the paperwork matches the number on the sterile shipment bag containing the tissue. The sterile shipment bag containing the tissue is then stored in a refrigerator until ready for further processing. All appropriate forms are completed and chain of custody and handling logs are also completed.

### Gross Tissue Processing Step

When the tissue is ready to be processed further, the sterile supplies necessary for processing the placenta tissue further are assembled in a staging area in a controlled environment and are prepared for introduction into a critical environment. If the critical environment is a manufacturing hood, the sterile supplies are opened and placed into the hood using conventional sterile techniques. If the critical environment is a clean room, the sterile supplies are opened and placed on a cart covered by a sterile drape. All the work surfaces are covered by a piece of sterile drape using conventional sterile techniques, and the sterile supplies and the processing equipment are placed on to the sterile drape, again using conventional sterile techniques.

If the placental tissue is collected prior to the completion or obtaining of results from the screening tests and delivery cultures, such tissue is labeled and kept in quarantine. The tissue is approved for further processing only after the required screening assessments and delivery cultures, which declare the tissue safe for handling and use, are satisfied.

Processing equipment is decontaminated according to conventional and industry-approved decontamination procedures and then introduced into the critical environment. The equipment is strategically placed within the critical environment to minimize the chance for the equipment to come in proximity to or be inadvertently contaminated by the tissue specimen.

Next, the placenta is removed from the sterile shipment bag and transferred aseptically to a sterile processing basin within the critical environment. The sterile basin contains, preferably, sterile water that is at room or near room temperature. The placenta is gently massaged to help separate blood clots and to allow the placenta tissue to reach room temperature. After having warmed up to the ambient temperature (after about 10-30 minutes), the placenta is then removed from the sterile processing basin and laid flat on a processing tray with the amniotic membrane layer facing down for inspection.

The placenta tissue is examined and the results of the examination are documented on a “Raw Tissue Assessment Form.” The placenta tissue is examined for discoloration, debris or other contamination, odor, and signs of damage. The size of the tissue is also noted. A determination is made, at this point, as to whether the tissue is acceptable for further processing.

Next, if the placenta tissue is deemed acceptable for further processing, the placental disc can be separated from the amnion, chorion, umbilical cord and other placental tissues. These other placental tissues are reserved and can be used to make additional products. The placental disc tissue is cut into pieces with dimensions of approximately 4 cm×4 cm×4 cm or smaller.

The cut placental disc tissue is transferred to a mixing flask. The desired amount of sterile water is poured into the mixing flask. The flask containing the placental disc tissue and the sterile water is then placed on a shaker platform, where it is agitated for a sufficient period of time, dependent upon the type of equipment available. After the placental disc tissue has been agitated, it is then removed from the shaker platform. The contents of the flask are then poured onto a strainer.

A sterile scoop is used to swirl the tissue in the strainer until most of the liquid has passed through. The placental disc tissue mixture continues to be poured from the flask into the strainer until all of the liquid has been completely drained.

The placental disc tissue is then transferred from the strainer into a blender. The placental disc tissue is then blended until the desired consistency is obtained. Once blending has been completed, sterile forceps can be used to further mix the tissue and dislodge any tissue strands that may stick to the sides of the blender or the blade. The placental disc tissue is then blended again. Once the blending process has been completed, the placental disc tissue mixture is poured into a flask. The appropriate amount of sterile water is added to the flask to obtain the desired volume. The flask is placed on the shaker platform

The placental disc tissue is subjected to another cycle of agitation until the mixture is adequately distributed. Upon completion of the agitation cycle, the flask is removed from the shaker platform.

The flask containing the placental disc tissue mixture is returned to the shaker platform, where it is agitated at a speed and time determined to adequately cleanse the tissue mixture. Once the agitation cycle has been completed, the placental disc tissue is poured into the strainer until most of the liquid has passed through. If blood clots are visible in the strained liquid, they are removed at this point. The placental disc tissue is returned back to the flask.

The cycle of adding water to the placental disc tissue in the flask, agitating it, straining it, and returning it to the flask is should be completed twice more before moving on to the decellularization steps.

### Decellularization Step

The contents of the flask are poured onto the strainer until the liquid level is near the top of the strainer. A sterile scoop is then used to swirl the tissue in the strainer until most of the liquid has passed through. The tissue is then collected from the strainer and transferred back to the flask. The appropriate amount of decellularization reagent is poured into the flask containing the placental disc tissue mixture. There are a variety of reagents and techniques that may be used in the process of tissue decellularization that are well-known to those skilled in the art. Specific reagents and procedures that may be used to obtain adequate decellularization of tissue are described in detail in U.S. Pat. Nos. 11,191,788; 7,723,108; 7,354,749; and 9,913,705 which are hereby incorporated by reference in their entireties. The decellularization process described herein is exemplary and is not meant to be limiting. The flask containing the decellularization reagent and the placental disc tissue is then placed on the shaker platform. The flask is then agitated at the appropriate speed and for a sufficient length of time for the decellularization reagent to be evenly distributed throughout the tissue. Once agitation has been completed, the contents of the flask are then poured onto the strainer until the liquid level is near the top of the strainer. Using a sterile scoop, the tissue in the strainer is swirled until most of the liquid has passed through. This process is continued until all of the liquid has been drained. The sterile scoop is then used to transfer the tissue back to the flask.

The appropriate amount of decellularization reagent is poured into the flask containing the placental disc tissue mixture. The flask containing the collected tissue is then transferred to a shaker platform. The flask is then agitated at the appropriate speed and for a period of time necessary for the decellularization reagent to completely dissolve the cells in the tissue. Once the agitation cycle has been completed, the tissue in the flask is poured into the strainer. A sterile scoop is then used to swirl the tissue in the strainer until most of the liquid has passed through. The sterile scoop is then used to transfer the strained tissue back into the flask. The appropriate amount of decellularization reagent is poured into the flask containing the placental disc tissue mixture. The flask is then placed on a shaker platform, where it is agitated at the desired speed and for the period of time necessary for adequate decellularization to occur. Once the agitation cycle has been completed, the contents of the flask are again poured onto the strainer. Pouring is stopped when the liquid level is near the top of the strainer. Using the sterile scoop, the tissue in the strainer is swirled until all of the liquid has been drained. The tissue is then collected using the sterile scoop and transferred back into the flask.

Next, the appropriate amount of decellularization reagent is poured into the flask containing the tissue. The flask is then placed on a shaker platform and agitated at the desired speed and the period of time necessary to achieve adequate mixing and decellularization of the tissue. Following the completion of the agitation cycle, the flask is removed from the shaker platform. The contents of the flask are then poured onto the strainer until the liquid level nears the top of the strainer. A sterile scoop is then used to swirl the tissue in the strainer until most of the liquid has passed through. The contents of the flask are completely emptied into the strainer until all of the liquid has been completely drained. The sterile scoop is then used to transfer the strained tissue back to the flask, where sterile water is added to the tissue in the flask. The flask with the sterile water and the strained tissue is then placed onto a shaker platform, where it is agitated at the speed and for the period of time necessary to adequately rinse the mixture. The decellularized PCM is then subjected to a series of additional rinse cycles, where water is added to the PCM in the flask, the flask is agitated, the PCM and water are strained, and the strained PCM is returned to the flask.

### Morselization Step

The wet tissue is then poured into a beaker and the total volume of the tissue is recorded. The wet tissue is then morselized by any suitable method known in the art. This includes blending, sonication, cryo-milling, cryo-fracturing or any other morselization techniques that would be known to those having skill in the art. For morselization via blending, the tissue is then transferred to a blender. Sterile water may be added at a volume greater than, equal to, or less than the volume of the tissue that was recorded in the beaker, based on desired density, thickness, absorptiveness, etc. of the final product. However, other carriers may be substituted for sterile water during this step, such as antibiotic solutions, glycol, etc. The tissue and the sterile water are blended based on the desired level of homogeneity of the sheet. The blended tissue is then poured into a beaker. Next, the blended tissue is put into a container having a desired shape. In an embodiment, the blended tissue is poured into a tray. The tray may be gently shaken to ensure even dispersal of the tissue across the surface of the tray.

### Dehydration Step

The tissue is then dehydrated. Dehydration may occur by any method known in the art, such as lyophilization, thermal dehydration, and chemical dehydration. In an embodiment, the blended tissue is lyophilized. In this embodiment, the tray containing the blended tissue is then placed into a Tyvek brand pouch and placed into a freezer. The frozen tissue contained within the Tyvek pouch is transferred to a commercially available lyophilizer device. The tissue is dehydrated using a low temperature process that involves freezing the product and lowering pressure, removing the ice by sublimation. The residual moisture content of the tissue following completion of the cycle is less than 15%.

### Cutting and Packaging Step

Upon completion of the lyophilization step, the Tyvek pouch containing the tissue is opened. The tissue sheet is then visually inspected to ensure uniformity. The sheet can also be cut and modified during this step, if desired. Last, the finished sheet is placed into a heat-sealed pouch for final packaging. The heat-sealed pouch may be sealed with ambient air, nitrogen, glycerol or other preservatives that are generally regarded as safe inside the packaging. Additionally, silicone backing may be cut according to the shape of the sheet and adhered to the sheet prior to placing it into the heat-sealed pouch.

### Overview of Clinical Applications

In practice, it has been determined that the above placental collagen sheet can be stored in room temperature conditions safely for at least five (5) years. Placental collagen sheet has been shown to be suitable for the following surgical procedures and indications: Surgical debridement, partial and full thickness wounds, diabetic foot ulcers (DFUs), venous leg ulcers (VLUs), pressure ulcers (PUs), chronic vascular ulcers, trauma wounds (abrasions, lacerations, burns, and skin tears), and draining wounds.

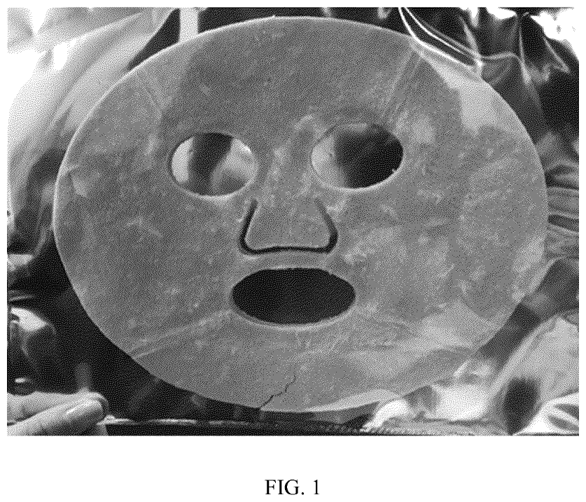

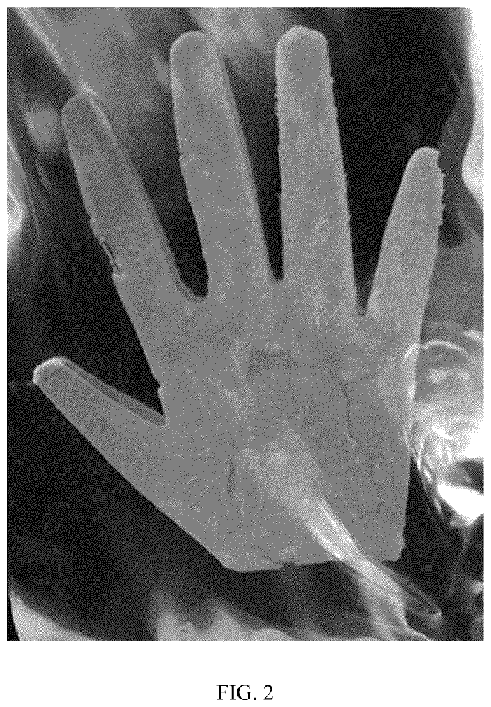

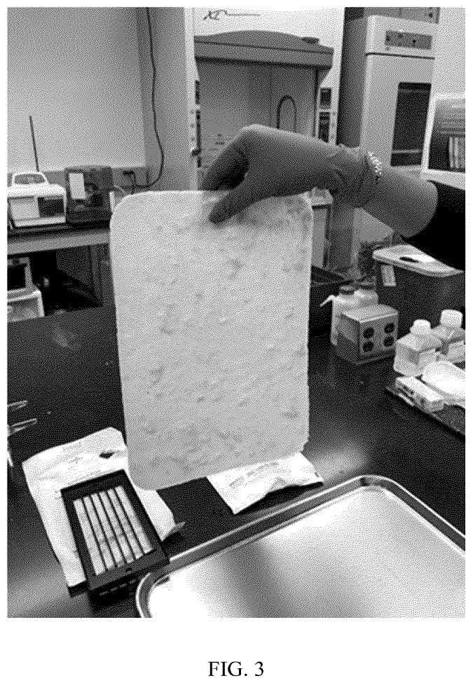

One particular advantage of the placental collagen sheet configuration is that custom sized trays may be used to create placental collagen sheets of varying shapes and sizes, that are not constrained by the size and shape of the source tissue. For example, it is possible to create a placental collagen sheet in the shape of a human face (FIG. 1), a human hand (FIG. 2) or a traditional rectangular sheet configuration (FIG. 3). These shapes are exemplary, and the sheet shape is only constrained by the shape of the tray in which the placental collagen sheet is placed into prior to lyophilization. It is also possible to precision cut the sheet into the desired shape by using a laser, blade, or other precision cutting tool. The custom placental collagen sheet is placed over the patient's wound area, and the sheet is then re-hydrated by soaking it in buffered saline solution (BSS), 0.9% saline solution, or sterile water for 30-90 seconds. The consistency of the sheet can be adjusted based on the soak time. For example, the sheet can be soaked for 15-30 seconds if a more malleable, sheet-like consistency is desired. Alternatively, soaking the sheet for a longer period, such as four to five minutes, will result in a putty-like consistency.

In addition, although the above procedures and tissues have been described for use with allograft tissues, such procedures and techniques are likewise suitable and usable for xenograft and isograft applications. In view of the foregoing detailed description of preferred embodiments of the present invention, it readily will be understood by those persons skilled in the art that the present invention is susceptible to broad utility and application. While various aspects have been described in the context of screen shots, additional aspects, features, and methodologies of the present invention will be readily discernible therefrom. Many embodiments and adaptations of the present invention other than those herein described, as well as many variations, modifications, and equivalent arrangements and methodologies, will be apparent from or reasonably suggested by the present invention and the foregoing description thereof, without departing from the substance or scope of the present invention. Furthermore, any sequence(s) and/or temporal order of steps of various processes described and claimed herein are those considered to be the best mode contemplated for carrying out the present invention. It should also be understood that, although steps of various processes may be shown and described as being in a preferred sequence or temporal order, the steps of any such processes are not limited to being carried out in any particular sequence or order, absent a specific indication of such to achieve a particular intended result. In most cases, the steps of such processes may be carried out in various different sequences and orders, while still falling within the scope of the present inventions. In addition, some steps may be carried out simultaneously. Accordingly, while the present invention has been described herein in detail in relation to preferred embodiments, it is to be understood that this disclosure is only illustrative and exemplary of the present invention and is made merely for purposes of providing a full and enabling disclosure of the invention. The foregoing disclosure is not intended nor is to be construed to limit the present invention or otherwise to exclude any such other embodiments, adaptations, variations, modifications and equivalent arrangements, the present invention being limited only by the claims appended hereto and the equivalents thereof.

### Alternative Embodiments Utilizing Umbilical Cord, Amnion, Chorion, or a Combination Thereof

Umbilical cord tissue may be substituted in favor of decellularized PCM by following an alternative processing method described in detail in U.S. Pat. App. Pub. No. US 2021/0393852, the contents of which are incorporated herein by reference in their entirety. The process described therein in the “Methods of Manufacture” section should be followed until the lyophilization step has been reached. At that point in the process, the umbilical cord tissue should be processed according to the morselization process set forth herein and to all subsequent processing steps until completion of the process has been achieved.

Amnion and/or chorion tissue may be substituted in favor of decellularized PCM by following an alternative processing method described in detail in U.S. Pat. App. Pub. No. US 2022/0133955, the contents of which are incorporated herein by reference in their entirety. The process described therein in the “Methods of Manufacture” section should be followed until the lyophilization step has been reached. At that point in the process, the amnion and/or chorion tissue should be processed according to the morselization process set forth herein and to all subsequent processing steps until completion of the process has been achieved.

What is claimed is:

1. A flowable composition, comprising decellularized, morselized placental disc and a carrier.

2. The composition of claim 1, wherein the composition has a viscosity of at most about 100,000 cP.

3. The composition of claim 2, wherein the composition has a viscosity of between about 0.89 cP and about 100,000 cP.

4. The composition of any one of claims 1-3, wherein the carrier is sterile water.

5. The composition of any one of claims 1-4, wherein the composition comprises about 70% to about 30% weight percent placental disc and about 30% to about 70% weight percent carrier.

6. The composition of any one of claims 1-5, wherein the composition does not comprise amnion, chorion, or umbilical cord.

7. The composition of any one of claims 1-6, wherein the composition consists essentially of decellularized, morselized placental disc and a carrier.

8. The composition of any one of claims 1-6, wherein the composition consists of decellularized, morselized placental disc and a carrier.

9. A dehydrated sheet comprising decellularized, morselized placental disc.

10. The sheet of claim 9, wherein the sheet has a thickness of at least about 0.05 mm.

11. The sheet of claim 10, wherein the sheet has a thickness of between about 0.05 mm and about 40 mm.

12. The sheet of any one of claims 9-11, wherein the sheet comprises a first side and a second side, and the first side has a surface area of at least about 0.5 cm².

13. The sheet of claim 12, wherein the surface area of said first side is between about 0.5 cm² and about 800 cm².

14. The sheet of any one of claims 9-13, wherein the sheet has not been treated with a crosslinking agent.

15. The sheet of any one of claims 9-14, wherein the sheet consists essentially of decellularized, morselized placental disc.

16. The sheet of any one of claims 9-14, wherein the sheet consists of decellularized morselized placental disc.

17. A sealed container comprising the sheet of any one of claims 9-16.

18. The sealed container of claim 17, wherein the container is deoxygenated.

19. The sealed container of claim 17 or 18, wherein the container is a sealed pouch.

20. A method of preparing a decellularized, morselized placental disc composition, said method comprising:
i) morselizing placental disc; and
ii) decellularizing the morselized placental disc obtained in step i).

21. The method of claim 20, further comprising an additional morselization step following step ii).

22. The method of any one of claims 20-21, further comprising
iii) transferring the decellularized, morselized placental disc composition into a container having a desired shape; and
iv) dehydrating the composition in the container,
thereby obtaining a dehydrated, decellularized, morselized placental disc sheet.

23. The method of claim 22, wherein said transferring is via pouring and said container is a tray or mold.

24. The method of claim 22, wherein said transferring is via injection and said container is a mold.

25. A flowable composition, comprising decellularized, morselized umbilical cord and a carrier.

26. A flowable composition, comprising decellularized, morselized amnion, chorion, umbilical cord, or any combination thereof, and a carrier.
